(19) United States
(12) Patent Application Publication
(10) Pub. No.: US 2010/0012848 A1
(43) Pub. Date: Jan. 21, 2010

(54) Obturator for real-time verification in gamma guided stereotactic localization

(75) Inventor: Benjamin L. Welch, Hampton, VA (US)

Correspondence Address: AUZVILLE JACKSON, JR., 8652 RIO GRANDE ROAD, RICHMOND, VA, 23229, US

(73) Assignee: Dilon Technologies, Inc.

(21) Appl. No.: 12/218,601

(22) Filed: Jul. 16, 2008

(51) Int. Cl. G01T 1/166; A61B 6/00

(52) U.S. Cl. 250/363.04; 382/131

(57) ABSTRACT

A combination rigid imager and obturator for real-time localization of a region of interest during the performance of a surgical procedure that includes: a) a rigid imaging grid preferably made up of an array of gamma radiation position sensitive photomultipliers; and b) an obturator inserted through the rigid imaging grid into a mass containing a region of interest and including: 1) an outer tube having a first closed end and a distal open end; and 2) an inner tube having an inserted end that includes a radioactive tracer and a remote end inserted into the distal open end; wherein the first closed end is inserted into the mass containing a region of interest and the radioactive source provides a marker for the region of interest during the performance of a surgical procedure.

### FIELD OF THE INVENTION

The present invention relates to gamma guided stereotactic imaging and more particularly to an obturator device for verifying image location and to a method of use thereof.

### BACKGROUND OF THE INVENTION

Gamma guided stereotactic imaging/localization uses two gamma camera images of an object taken at different angles to determine the three dimensional location of the region of interest in that object. The location can be used, for example, for positioning a needle in a suspected tumor to collect a tissue sample for biopsy. In some situations, it is desirable to have real-time verification of the location during the procedure. This requires that a marker be placed at the location of the region of interest. In order to be imaged by the gamma camera, the marker must be radioactive, and capable of removal without leaving contamination and should be seen in the image at the time of verification.

Thus, there is a need for a system or method and device for providing such a marker for purposes of real-time localization during gamma imaging.

### OBJECT OF THE INVENTION

It is therefore an object of the present invention to provide both a device and a method for the provision of real-time localization information during stereotactic gamma imaging.

### SUMMARY OF THE INVENTION

The present invention provides a combination rigid localization grid, stereotactic gamma imager, and an obturator for real-time localization of a region of interest during the performance of a surgical procedure that includes: a) a rigid grid positioning system preferably made up of coordinate grid on an (x, y) translation stage; b) a stereotactic gamma imager preferably comprising a stereo viewing collimator and a gamma imager; and c) an obturator inserted through the rigid grid positioning system into a mass containing a region of interest and including: 1) an outer tube having a first closed end and a distal open end; and 2) an inner tube having an inserted end that includes a radioactive tracer and a remote end inserted into the distal open end; wherein the first closed end is inserted into the mass containing a region of interest and the radioactive source provides a marker for the region of interest during the performance of a surgical procedure.

### DESCRIPTION OF THE DRAWINGS

FIG. 1 is a cross-sectional view of the inner tube of the obturator of the present invention.

FIG. 2 is a cross-sectional view of the outer tube of the obturator of the present invention.

FIG. 3 is a cross-sectional view of the obturator of the present invention wherein the inner tube containing a radioactive marker is inserted into the outer tube in accordance with the present invention.

FIG. 4 is a top plan view of the obturator of the present invention being used in conjunction with a grid positioning system.

FIG. 5 is a cross-sectional view of the procedure depicted in FIG. 4 showing the obturator used in conjunction with a grid positioning and stereotactic gamma imager.

### DETAILED DESCRIPTION

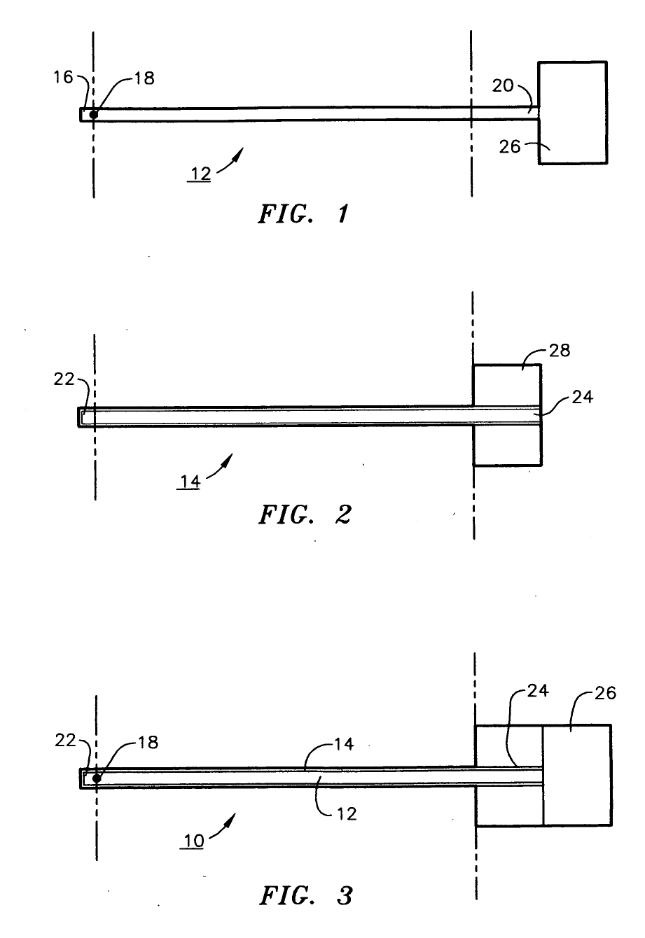

Referring now to the accompanying drawings, the obturator 10 of the present invention (best seen as a complete assembly in FIG. 3), comprises an inner tube 12 (see particularly FIG. 1) and a sterile outer tube 14 (see particularly FIG. 2). As seen in FIG. 3, in the assembled configuration, inner tube 12 is inserted into outer tube 14. Inner tube 12 has an inserted end 16 that includes a radioactive source or marker 18 and a distal end 20. Outer tube 14 has a closed end 22 and a remote end 24 that is open and through which inner tube 12 containing radioactive marker 18 is inserted. A variety of fittings 26 and 28 may be attached to inner and outer tubes 12 and 14 for handling, attachment, assembly etc. purposes.

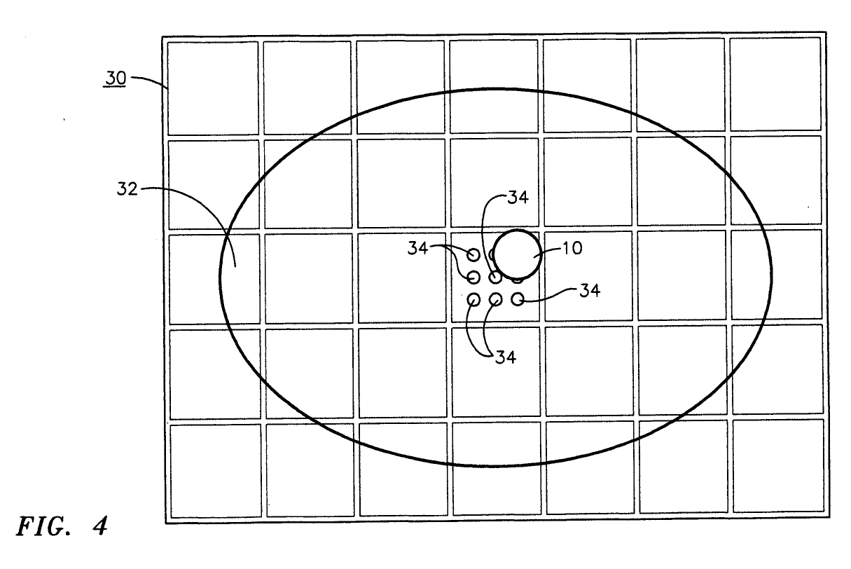

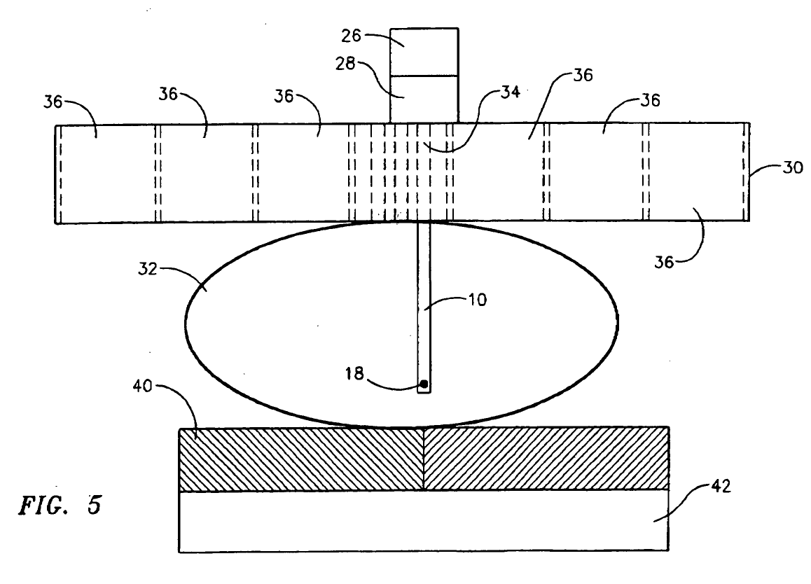

As seen in FIGS. 4 and 5, in use in accordance with a preferred embodiment of the present invention, obturator 10 is used in concert with a rigid grid positioning system 30, and a stereotactic gamma imager (40 and 42) that is used to view a mass 32 containing a particular region of interest. As seen in FIG. 4, obturator 10 is inserted through an aperture 34 in localization grid 30, which is mechanically registered with a stereotactic imager preferably comprising a stereo viewing collimator system (40) composed of a pair of slant hole collimators and a gamma imager (42) composed of position sensitive photomultipliers such as are well known and widely described in the prior art. As best seen in FIG. 5, after insertion through stereotactic gamma imager 30 closed end 22 is inserted into mass 32 that contains the region of interest and radioactive source 18 is present in the region of interest during the imaging using stereotactic imager (40 and 42).

In practical use, obturator 10 is assembled as shown in FIG. 3. Grid positioning system 30 and stereotactic gamma imager 40/42 are placed adjacent to mass 32 and images of mass 32 acquired. The locations of the regions of interest in mass 32 observed in these thus acquired images are determined and their spatial positions (X, Y and Z locations) calculated. An incision is then made in mass 32 to the calculated location of the region of interest based upon the previously acquired image(s). Obturator 10 is then inserted into the opening created by the incision to the required depth. The obturator can then be imaged to determine if first end 16 containing radioactive source 18 is in the correct position, i.e. at the location of the region of interest as determined from the previously acquired image(s). Once it has been assured that the appropriate region has been precisely located, obturator 10 is removed and a desired procedure such as insertion of a biopsy needle and removal of tissue samples performed.

There has thus been described an obturator and method of use thereof that permits real-time localization of an area of interest during stereotactic gamma imaging.

As the invention has been described, it will be apparent to those skilled in the art that the same may be varied in many ways without departing from the spirit and scope of the invention. Any and all such modifications are intended to be included within the scope of the appended claims.

What is claimed is:

1) In combination, a rigid localization, stereotactic gamma imager and an obturator for real-time localization of a region of interest during the performance of a surgical procedure comprising:
a) a rigid localization grid including;
b) a stereotactic gamma imager for the acquisition of images useful in calculating the spatial location of a region of interest in a mass under study; and
c) an obturator inserted through the rigid localization grid into the mass and comprising:
i) an outer tube having a first closed end and a distal open end; and
ii) an inner tube having an inserted end that includes a radioactive tracer and a remote end inserted into the distal open end;
wherein the first closed end is inserted into the mass containing a region of interest and the radioactive source provides a marker for the region of interest during subsequent imaging and thereby specific localization of the region of interest for subsequent procedures.

2) The combination of claim 1 wherein the rigid localization grid is mechanically co-registered to a stereotactic gamma imager which comprises a set of stereo viewing slant-hole collimators and an array of gamma radiation sensitive crystals and position sensitive photomultipliers.

3) A method for real-time imaging and localization of a region of interest in a mass under study comprising exposing the mass using a combination stereotactic gamma imager and an obturator for real-time localization of the region of interest comprising:
a) a stereotactic gamma imager comprising an array of gamma radiation position sensitive photomultipliers; and
b) an obturator inserted through the array of gamma radiation position sensitive photomultipliers into a mass containing a region of interest and comprising:
i) an outer tube having a first closed end and a distal open end; and
ii) an inner tube having an inserted end that includes a radioactive tracer and a remote end inserted into the distal open end;
wherein the first closed end is inserted into the mass containing a region of interest and the radioactive source provides a marker for the region of for subsequent procedures.

4) An obturator comprising:
i) an outer tube having a first closed end and a distal open end; and
ii) an inner tube having an inserted end that includes a radioactive tracer and a remote end inserted into the distal open end.
